(19) United States
(12) Patent Application Publication
(10) Pub. No.: US 2011/0269162 A1
(43) Pub. Date: Nov. 3, 2011

(54) UBIQUITIN PROTEASOME SYSTEM PROFILING FOR DIAGNOSIS OF CHRONIC LIVER DISEASE

(75) Inventors: Maher Albitar, Coto de Caza, CA (US); Wanlong Ma, (US); Ke Zhang, (US); Xi Zhang, (US); Xiuqiang Wang, (US); Kevin Qu, (US); Anthony Sferruzza, (US)

(73) Assignee: Quest Diagnostics Investments Incorporated

(21) Appl. No.: 12/912,600

(22) Filed: Oct. 26, 2010

Related U.S. Application Data
(60) Provisional application No. 61/256,251, filed on Oct. 29, 2009.

(51) Int. Cl. C12Q 1/37; C12Q 1/42; C12Q 1/52

(52) U.S. Cl. 435/16; 435/23; 435/21

(57) ABSTRACT

Provided herein are methods for the diagnosis, staging, prognosis, or management of liver disease, e.g. chronic liver disease, and other diseases using profiles of the ubiquitin-proteasome system determined from acellular body fluids or cell-containing samples. Further provided are methods of predicting response to therapy in certain populations of patients with liver disease.

### CROSS-REFERENCE TO RELATED APPLICATIONS

This application claims priority to U.S. Provisional App. No. 61/256,251, entitled “UBIQUITIN PROTEASOME SYSTEM PROFILING FOR DIAGNOSIS OF CHRONIC LIVER DISEASE”, filed Oct. 29, 2010 which is incorporated herein by reference in its entirety and for all purposes.

### FIELD OF THE INVENTION

The invention relates to the diagnosis, prognosis, and management of disease, including liver disease.

### BACKGROUND OF THE INVENTION

The following discussion of the background of the invention is merely provided to aid the reader in understanding the invention and is not admitted to describe or constitute prior art to the present invention.

The ubiquitin-proteasome system (UPS) is responsible for the degradation of approximately 80-90% of normal and abnormal intracellular proteins and therefore plays a central role in a large number of physiological processes. For example, the regulated proteolysis of cell cycle proteins, including cyclins, cyclin-dependent kinase inhibitors, and tumor suppressor proteins, is required for controlled cell cycle progression and proteolysis of these proteins occurs via the ubiquitin-proteasome pathway (Deshaies, Trends in Cell Biol., 5:428-434 (1995) and Hoyt. Cell, 91:149-151 (1997)). In another example, the activation of the transcription factor NF-κB, which itself plays a central role in the regulation of genes involved in the immune and inflammatory responses, is dependent upon the proteasome-mediated degradation of an inhibitory protein. Iκα B-α (Palombella et al., WO 95/25533). In yet another example, the ubiquitin-proteasome pathway plays an essential role in antigen presentation through the continual turnover of cellular proteins (Goldberg and Rock, WO 94/17816).

While serving a central role in normal cellular homeostasis, the UPS also mediates the inappropriate or accelerated protein degradation occurring as a result or cause of pathological conditions including cancer, inflammatory diseases, and autoimmune diseases, characterized by deregulation of normal cellular processes. Central to this system is the 26S proteasome, a multi-subunit proteolytic complex, consisting of one 20S proteasome core and two flanking 19S complexes. The 20S proteasome consists of four rings: two outer α-rings and two inner β-rings surrounding a barrel-shaped cavity. The two inner β-rings form a central chamber that harbors the catalytic site for the chymotryptic, tryptic, and caspase-like activities (von Mikecz, J Cell Sci, 119(10):1977-84, 2006).

Proteins targeted for degradation by the proteasome contain a recognition signal. This signal consists of a polyubiquitin chain that is selectively attached to the protein target by the sequential addition of ubiquitin monomers. The polyubiquitin signal is recognized by the 19S complex, which mediates the entry of the target protein into the proteolytic chamber.

### SUMMARY OF THE INVENTION

The present invention is based on the discovery that the specific activity of proteasomal peptidases may be detected in patient samples and that such activity can have clinical value in the diagnosis and prognosis of certain disease states.

In one aspect, the invention provides methods for diagnosing chronic liver disease (e.g., HCV-related CLD) in a subject, the method comprising: determining, in a body fluid sample (e.g., an acellular body fluid sample) obtained From the subject, a single diagnostic score using the specific activity of one or more (i.e., one, two, or three) proteasomal peptidases selected from the group consisting of chymotrypsin-like activity (Ch-L), trypsin-like activity (Tr-L), and caspase-like activity (Cas-L), wherein the specific activity is determined by normalizing the one or more peptidase activities to the amount of proteasomal protein in the sample, and wherein a difference of the specific activity of one or more proteasmal peptidases compared to a reference specific activity (e.g., a reference score) indicates a liver disease in the subject. In one embodiment, the acellular body fluid is selected from the group consisting of serum and plasma.

In one embodiment, the reference specific activity is the specific activity in a comparable sample from one or more healthy individuals. In one embodiment, the level of specific activity of one or more proteasomal peptidases is compared to a reference score determined from the level of specific activity of one or more proteasomal peptidases present in a comparable sample from healthy individuals, and wherein an increase or decrease in the subject value relative to the reference score is used to determine a diagnosis for the subject.

In one aspect, the present invention provides a method of diagnosing a chronic liver disease in a subject, the method comprising: determining the amount of proteasomal protein in a test sample for the subject; determining the level of one or more proteasomal peptidase activities in a test sample from the subject, the peptidase activities selected from the group consisting of chymotrypsin-like activity (Ch-L), trypsin-like activity (Tr-L), and caspase-like activity (Cas-L), normalizing the level of one or more proteasomal peptidase activities to the amount of proteasomal protein to provide a specific activity of the one or more proteasomal peptidases; determining a single diagnostic score using the specific activity of the one or more proteasomal peptidases; and comparing the diagnostic score to a reference score to allow for the diagnosis of the presence of a chronic liver disease in the subject.

In another aspect, the present invention provides a method for diagnosing chrome liver disease in a subject, the method comprising: (a) determining the amount of one or more of chymotrypsin-like activity (Ch-L), trypsin-like activity (Tr-L), and caspase-like activity (Cas-L) in a sample from the subject; (b) determining the amount of one or more of ALT, ASP, ALP, bilirubin, albumin, and ubiquitin in the sample; (c) determining the amount of proteasomal protein in the sample and normalizing one or more of Ch-L, Tr-L, and Cas-L to calculate the specific activity; (d) determining a single diagnostic score for the subject based on the results obtained in steps (b) and (c); and (e) comparing the diagnostic score to a reference score that is predictive of a disease or symptom in order to determine the presence of CLD in the subject.

In one embodiment, the amount of each of the Cas-L activity, Tr-L activity, and Ch-L activity are assayed in a sample from the subject. In one embodiment, the amount of at least one of ALT, ASP, and ALP are assayed in a sample from the subject. In one embodiment for the diagnosis of CLD, the score is determined using the algorithm:

Score=y/(1+y)

wherein,

y=exp [−X+(C₁×Age)+(C₂×Tr-L/p)+(C₃×Ubiquitin)+(C₄×ALT)+(C₅×ASP)+(C₆×ALP)]

wherein X is from −9.706 to −2.7958 inclusive; C₁ is from 0.0957 to 0.1835 inclusive; C₂ is from −0.1300 to −0.0578 inclusive; C₃ is from −0.0381 to −0.0143 inclusive; C₄ is from −0.1827 to −0.0965 inclusive; C₅ is from 0.2165 to 0.4107 inclusive: C₆ is from −0.0508 to −0.0222 inclusive; and wherein, age is provided in years; normalized Tr-L (Tr-L/p) is reported in pmol product /sec/pg proteasome; ubiquitin is reported in mg/dL; ASP, ALT, and ALP are reported in IU/L. In a particular embodiment, X is about −6.2540; C₁ is about 0.1396; C₂ is about −0.0939; C₃ is about −0.0262; C₄ is about −0.1396; C₃ is about 0.3136; and C₆ is about −0.0365.

In one embodiment, the reference score is about 0.5 and a score less than about 0.5 is indicative of the absence of CLD in the subject. In one embodiment, the reference score is about 0.5 and a score greater than or equal to about 0.5 is indicative of CLD in the subject. In one embodiment, the score is used for the choice of a suitable treatment for the subject.

In one aspect, the present invention provides a method for staging CLD and diagnosing advanced liver fibrosis in a subject, the method comprising: (a) determining the amount of one or more of chymotrypsin-like activity (Ch-L), trypsin-like activity (Tr-L), and caspase-like activity (Cas-L) in a sample from the subject; (b) determining the amount of one or more of ALT, ASP, ALP, bilirubin, albumin, and ubiquitin in the sample; (c) determining the amount of proteasomal protein in the sample and normalizing one or more of Ch-L, Tr-L, and Cas-L to calculate the specific activity; (d) determining a single diagnostic score for the subject based on the results obtained in steps (b) and (c); and (e) comparing the diagnostic score to a reference score that is predictive of a disease or symptom in order to determine the presence of advanced liver fibrosis in the subject.

In one embodiment, the amount of each of the Cas-L activity, Tr-L activity, and Ch-L activity are assayed in a sample from the subject. In one embodiment, the amount of at least one ALT, ASP, and ALP are assayed in a sample from the subject. In one embodiment for the staging of CLD and the diagnosis of advanced liver fibrosis, the score is determined using the algorithm:

Score=y/(1+y)

wherein,

y=exp [−X+(C₁×Age)+(C₂×Tr-L/p)+(C₃×ALT)+(C₄×Bilirubin)×(C₅×Albumin)]

wherein X is from 0.7274 to 16.3490 inclusive; C₁ is from 0.0368 to 0.1682 inclusive; C₂ is from 0.0494 to 0.1556 inclusive; C₃ is from 0.0173 to 0.0433 inclusive; C₄ is from 0.7512 to 3.9666 inclusive; and C₅ is from −6.4955 to −2.6163 inclusive; and wherein, age is provided in years; normalized Tr-L (Tr-L/p) is reported in pmol product /sec/pg proteasome; ALT is reported in IU/L; and bilirubin and ubiquitin are reported in mg/dL.

In a particular embodiment. X is about 8.5382; C₁ is about 0.1025; C₂ is about 0.1025; C₃ is about 0.0303; C₄ is about 2.3589; and C₅ is about −4.5559. In one embodiment, the reference score is about and and a score less than about 0.5 is indicative of the absence of advanced liver fibrosis in the subject. In one embodiment, the reference score is about 0.5 and a score greater than or equal to about 0.5 is indicative of advanced liver fibrosis in the subject.

### DETAILED DESCRIPTION

The present invention relates generally to methods of assessing the ubiquitin-proteasome system (UPS) for the diagnosis of disease. As demonstrated herein, increasing or decreasing amounts of the specific activity of one or more proteasomal peptidases correlates with the presence of disease or the prognosis of a patient suffering from a disease. In particular, methods for diagnosing or staging liver diseases, e.g., HCV-related CLD, determining the likelihood of survival, and methods for predicting likelihood for responsiveness to therapy are provided.

The present technology is described herein using several definitions, as set forth throughout the specification. As used herein, unless otherwise stated, the singular forms “a,” “an,” and “the” include plural reference. Thus, for example, a reference to “a proteasome” is a reference to one or more proteasomes.

The term “about” as used herein in reference to quantitative measurements or values, refers to the enumerated value plus or minus 10%, unless otherwise indicated.

The term “antibody” as used herein encompasses both monoclonal and polyclonal antibodies that fall within any antibody classes, e.g., IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The terms “assessing” and “evaluating” are used interchangeably to refer to any form of measurement, and include determining if a characteristic, trait, or feature is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample; for example, the sample may be frozen at about −20° C. to about −70° C. Suitable body fluids are acellular fluids. “Acellular” fluids include body fluid samples in which cells are absent or are present in such low amounts that the peptidase activity level determined reflects its level in the liquid portion of the sample, rather than in the cellular portion. Typically, an acellular body fluid contains no intact cells. Examples of acellular fluids include plasma or serum, or body fluids from which cells have been removed.

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes (e.g., liver enzymes), examination of blood cells or bone marrow cells, cytogenetics, and immunophenotyping of blood cells. Specific activity of one or more proteasomal peptidases is a clinical factor.

The term “comparable” or “corresponding” in the context of comparing two or more samples, means that the same type of sample (e.g., plasma) is used in the comparison. For example, a specific activity level of one or more proteasomal peptidases in a sample of plasma can be compared to a specific activity level in another plasma sample. In some embodiments, comparable samples may be obtained from the same individual at different times. In other embodiments, comparable samples may be obtained from different individuals (e.g., a patient and a healthy individual). In general, comparable samples are normalized by a common factor. For example, acellular body fluid samples are typically normalized by volume body fluid and cell-containing samples are normalized by protein content or cell count.

The phrase “reference score” as used herein refers to a UPS score that is statistically predictive of a symptom or disease or lack thereof. In a particular embodiment, the reference score is about 0.5 and the UPS score distinguishes between CLD and an absence of CLD. For example, a UPS score greater than or equal to a reference score of about 0.5 is predictive of CLD. A UPS score less than a reference score of about 0.5 is predictive of an absence of CLD. In certain embodiments, this reference score may be between 0.425 to 0.575 inclusive, or between 0.450 to 0.550 inclusive, or between 0.475 to 0.525 inclusive. Alternatively, the reference score may be 0.425, 0.450, 0.5 0.475, 0.525, 0.550, and even 0.575. The above numbers are subject to 5% variation.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical art fir a particular disease or disorder, e.g., a liver disease.

As used herein, the phrase “difference of the level” refers to differences in the quantity of a particular marker, such as a protein or protein activity, in a sample as compared to a control or reference level. For example, the quantity of particular protein and/or the amount of a protein activity may be present at an elevated amount or at a decreased amount in samples of patients with a liver disease compared to a reference level. In one embodiment, a “difference of a level” may be a difference between the specific activity of a proteasomal peptidase present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%. at least about 80% or more. In one embodiment, a “difference of a level” may be a statistically significant difference between the specific activity of a proteasomal peptidase present in a sample as compared to a control. For example, a difference may be statistically significant if the measured level of the specific activity falls outside of about 1.0 standard deviations, about 1.5 standard deviations, about 2.0 standard deviations, or about 2.5 stand deviations of the mean of any control or reference group.

The term “enzyme linked immunosorbent assay” (ELISA) as used herein refers to an antibody-based assay in which detection of the antigen of interest is accomplished via an enzymatic reaction producing a detectable signal. ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generated a detectable product upon digestion of appropriate substrates.

The term “label” as used herein, refers to any physical molecule directly or indirectly associated with a specific binding agent or antigen which provides a means for detection for that antibody or antigen. A “detectable label” as used herein refers any moiety used to achieve signal to measure the amount of complex formation between a target and a binding agent. These labels are detectable by spectroscopic, photochemical, biochemical, immunochemical, electromagnetic, radiochemical, or chemical means, such as fluorescence, chemifluoresence, or chemiluminescence, electrochemiluminescence or any other appropriate means. Suitable detectable labels include fluorescent dye molecules or fluorophores.

The term “liver disease” refers to and comprises all kinds of disorders that affect the anatomy, physiology, metabolism, and/or genetic activities of the liver, that affect the generation of new liver cells and/or the regeneration of the liver, as a whole or parts thereof, transiently, temporarily, chronically or permanently, in a pathological way. In some embodiments, the liver disease is caused by alcohol (e.g. ASH), non-alcoholic fatty liver changes (such as NAFLD including NASH), nutrition-mediated liver injury, other toxic liver injury (such as unspecific hepatitis induced by e.g. drugs such as but not limited to acetaminophen (paracetamol), chlorinated hydrocarbons (e.g. CCl₄), amiodarone (cordarone), valproate, tetracycline, iscmiaeid, or food intoxication resulting in acute or chronic liver failure, e.g. by consumption of mushrooms containing aflatoxins or ingestion of certain metals (such as copper or cadmium) or herbal products used in natural medicine (homeopoatics such as Milk thistle, Chaparral, Kawa-Kawa), interference of bilirubin metabolism, hepatitis like syndromes, cholestasis, granulomatous lesions, intrahepatic vascular lesions and cirrhosis), trauma and surgery, and radiation-mediated liver injury (such as caused by radiotherapy). In one embodiment, the liver disease is caused by an infection. e.g., by hepatitis B virus (HBV) and hepatitis C virus (HCV) infections, and autoimmune-mediated liver disease (e.g. autoimmune hepatitis). Further included is liver injury due to sepsis. In some embodiment, liver disease is further understood to comprise genetic liver disorders (such as heamo-chromatosis and alphal antitrypsin deficiency), and other inherited metabolic liver diseases, e.g. metabolic steatohepatitis (MSH).

The term “Metavir Score” refers to a system for grading liver biopsy specimens. This scoring system assigns two standardized numbers: one to represent the degree of inflammation (activity score) and the other the degree of fibrosis (fibrosis score). The fibrosis is traded on a 5-point scale from 0 to 4, where F0 represents very low or no fibrosis; F1, F2 and F3 represent intermediate fibrosis stages; and F4 represents severe fibrosis (Knodell et al., Hepatology 1:431-435 (1981)).

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a longer than expected life expectancy, and the like.

As used herein, “plasma” refers to acellular fluid found in blood. Plasma may be obtained from blood by removing whole cellular material from blood by methods known in the art (e.g., centrifugation, filtration, and the like). As used herein, “peripheral blood plasma” refers to plasma obtained from peripheral blood samples.

As used herein, “serum” includes the fraction of plasma obtained after plasma or blood is permitted to clot and the clotted fraction is removed.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, rihosylated forms, acetylated forms, uhiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

As used herein, the term “proteasome” refers to certain large protein complexes within cells or body fluid that degrade proteins that have been tagged for elimination, particularly those tagged by ubiquitination. Proteasomes degrade denatured, misfolded, damaged, or improperly translated proteins. Proteasomal degradation of certain proteins, such as cyclins and transcription factors, serves to regulate the levels of such proteins. Exemplary proteasomes include the 26S proteasome, 20S proteasome, and the immunoproteasome.

The “26S proteasome” consists of 3 subcomplexes. The 26S proteasome consists of a 20S proteasome at the core which is capped at each end by a 19S regulatory particle (RP or PA700). The 19S RP mediates the recognition of the ubiquitinated target proteins, the ATP-dependent unfolding and the opening of the channel in the 20S proteasome, allowing entry of the target protein into the proteolytic chamber.

The “20S proteasome,” which forms the core protease (CP) of the 26S proteasome, is a barrel-shaped complex consisting of four stacked rings, each ring having 7 distinct subunits. The four rings are stacked one on top of the other and are responsible for the proteolytic activity of the proteasome. There are two identical outer a rings, having no known function, and two inner β rings, containing multiple catalytic sites. In eukaryotes, two of these sites on the β rings have chymotrypsin-like activity (Ch-L), two of these sites have trypsin-like activity (Tr-L), and two have caspase-like activity (Cas-L).

The “immunoproteasome,” which is characterized by an ability to generate major histocompatibility complex class I-binding peptides, consists of a 20S proteasome core capped on one end by 19S RP and on the other end by PA28, an activator of the 20S proteasome and an alternative RP. PA28 consists of two homologous subunits (termed α and β) and a separate but related protein termed PA28γ (also known as the Ki antigen).

The term “proteasomal peptidase activity” refers to any proteolytic enzymatic activity associated with a proteasome, such as the 26S or 20S proteasomes. The peptidase activities of proteasomes include, for example, chymotrypsin-like activity (Ch-L), trypsin-like activity (Tr-L), and caspase-like activity (Cas-L). In some embodiments, proteasomal peptidase activity is determined by measuring the rate of cleavage of a substrate per unit volume of body fluid assayed. Thus, the activity may be expressed as (moles of product formed)/time/(volume body fluid). For example, the activity may be expressed as pmol/sec/mL.

As used herein, the term “reference level” refers to a level of a substance which may be of interest for comparative purposes. In one embodiment, a reference level may be the specific activity level of a proteasomal peptidase expressed as an average of the level of the specific activity of the proteasomal peptidase from samples taken from a control population of healthy (disease-free) subjects. In another embodiment, the reference level may be the level in the same subject at a different time, e.g., before the present assay such as the level determined prior to the subject developing the disease or prior to initiating therapy. In general, samples are normalized by a common factor. For example, acellular body fluid samples are normalized by volume body fluid and cell-containing samples are normalized by protein content or cell count.

As used herein, the term “sample” may include, but is not limited to, bodily tissue or a bodily fluid such as blood (or a fraction of blood such as plasma or serum), lymph, mucus, tears, saliva, sputum, urine, semen, stool, CSF, ascites fluid, or whole blood, and including biopsy samples of body tissue. A sample may be obtained from any subject, e.g., a subject/patient having or suspected to have a liver disease.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a liver disease.

As used herein, the term “specific activity” of One or more proteasomal peptidases refers to the proteasomal peptidase activity in the sample that is normalized relative to the proteasomal protein content in the sample. Specific activity of the chymotrypsin-like, trypsin-like, and caspase-like proteasomal peptidases may be designated Ch-L/p, Tr-L/p, or Cas-L/p, respectively. The skilled artisan understands that normalization of the proteasomal peptidase activity to the proteasomal protein content in the sample involves measuring and expressing the amount of proteasomal protein per unit volume of body fluid assayed, in the same type of sample (preferably a split sample) as used to measure enzymatic activity. For example, proteasomal protein may be expressed as picograms (pg) of protein per mL which, when used to normalize a proteasomal peptidase activity expressed in pmol/sec/mL, results in a specific activity expressed in pmol/sec/pg proteasomal protein.

The phrase “substantially the same as” in reference to a comparison of one value to another value for the purposes of clinical management of a disease or disorder means that the values are statistically not different. Differences between the values can vary, for example, one value may be within 20%, within 10%, or within 5% of the other value.

As used herein, the term “a single diagnostic score” refers to a single number or score that is calculated from or, based on a statistical analysis of, the measured level of a plurality of biomarkers. The single diagnostic score determined from such an analysis of a sample obtained from a subject in a condition or set of conditions, e.g. a pathology such as chronic liver disease, can then he compared to another single diagnostic score determined from such an analysis of another sample obtained from a subject in a condition or set of conditions, such as a healthy individual. As used herein, the term “a single reference score” refers to a single diagnostic Score determined from the analysis of one or more reference samples, such as from one or more healthy individuals. As used herein, the term “UPS Score” refers to a specific type of single diagnostic score, based on a statistical analysis of the measured level of one or more biomarkers including, but not limited to, Ch-L/p, Cas-L/p, and Tr-L/p, that reflects a relationship of a specific subject to any one particular group of individuals, such as normal individuals or individuals having a disease or any progressive state thereof. In some embodiments, the UPS score is derived from a quantitative multivariate analysis, which reflects the overall statistical assessment of an individual patient's clinical condition based upon an integrated statistical calculation of a plurality of qualitatively unique factors, e.g., specific activity of proteasomal peptidases, proteasome level, age, gender, etc.

### Overview

Disclosed herein are methods for detecting the presence or absence of chronic liver disease (CLD) in subjects based, at least in part, on results of testing methods of the present technology on a sample. Further disclosed herein are methods for monitoring the status of subjects diagnosed with CLD based at least partially on results of tests on a sample. The test samples disclosed herein are represented by, but not limited in anyway to, sputum, blood (or a fraction of blood such as plasma, serum, or particular cell fractions), lymph, mucus, tears, saliva, urine, semen, ascites fluid, whole blood, and biopsy samples of body tissue. This disclosure is drawn, inter alia, to methods of diagnosing and monitoring liver diseases using profiles of the ubiquitin-proteasome system (UPS).

Biopsy is considered the gold standard for assessment of liver disease, but is an invasive procedure that carries the risk of complications. Moreover, biopsy can yield misleading results when a representative sample is not obtained. Alternative tests that are simple, reliable, and noninvasive would thus be of benefit in the diagnosis and staging of CLD. A link has been established between alterations in the ubiquitin-proteasome system (UPS) and CLD. The UPS is a major non-lysosomal proteolytic system in cells and plays a major role in regulating most cellular functions, including cell cycle regulation, apoptosis, differentiation, and DNA repair. Proteasomes from different tissues or cell types have different enzymatic activity patterns and molecular compositions. Alterations in any of the key UPS functions, including proliferation and apoptosis, can lead to hepatocellular injury.

In one aspect, the present invention relates to using a “UPS signature profile” in the diagnosis of CLD and a healthy control population. The UPS signature profile may be used alone or combined with other liver function tests, for assessment of liver fibrosis in patients with CLD, including, HCV-related CLD. The ubiquitin-proteasome system (UPS) plays a major role in the most important processes that control cell homeostasis in normal and disease states. The present inventors have discovered that analyzing various components of the UPS can provide a profile that may be used for classifying and stratifying CLD patients for diagnosis, therapy, and prediction of clinical behavior.

In various embodiments, the present methods overcome problems of CLD diagnosis and staging by determining the levels of proteasomes and proteasomal peptidase activities in the plasma of patients having or suspected of having liver diseases. By studying the levels of proteasome, ubiquitin, and proteasome enzymatic activities in the plasma, a UPS profile of the CLD can be determined. The use of UPS profiles in diagnosing and staging CLD is described in further detail below and in the Examples.

In one aspect, the methods generally provide for the detection, measuring, and comparison of a pattern of UPS proteins and/or activities in a patient sample. Additional diagnostic markers may be combined with the UPS profile to construct models for predicting the presence or absence or stage of a disease. For example, clinical factors of relevance to the diagnosis of CLD diseases, include, but are not limited to, the patient's medical history, a physical examination, complete blood count, the level of liver enzymes ALT, ALP and AST, and other markers. Moreover, biomarkers relevant to a particular liver disease may be combined with a subject's UPS profile to diagnose a disease or condition.

Accordingly, the various aspects relate to the collection, preparation, separation, identification, characterization, and comparison of the abundance of UPS proteins and/or activities in a test sample. The technology further relates to detecting and/or monitoring a sample containing one or more UPS proteins or activities, which are useful, alone or in combination, to determine the presence or absence of a liver disease or any progressive state thereof.

### Sample Preparation

Test samples of acellular body fluid or cell-containing samples may be obtained from an individual or patient. Methods of obtaining test samples are well-known to those of skill in the art and include, but are not limited to, aspirations or drawing of blood or other fluids. Samples may include, but are not limited to, whole blood, serum, plasma, saliva, cerebrospinal fluid (CSF), pericardial fluid, pleural fluid, urine, and eye fluid.

In embodiments in which the proteasome activity will be determined using an acellular body fluid, the test sample may be a cell-containing liquid or an acellular body fluid (e.g., plasma or serum). In some embodiments in which the test sample contains cells, the cells may be removed from the liquid portion of the sample by methods known in the art (e.g., centrifugation) to yield acellular body fluid for the proteasome activity measurement. In suitable embodiments, serum or plasma are used as the acellular body fluid sample. Plasma and serum can be prepared from whole blood using suitable methods well-known in the art. In these embodiments, data may be normalized by volume of acellular body fluid.

In some embodiments, the proteasomal peptidase activity is determined using a cell-containing sample. In these embodiments, the cell-containing sample includes, but is not limited to, blood, urine, organ, and tissue samples. In suitable embodiments, the cell-containing sample is a blood sample, such as a blood cell lysate. Cell lysis may be accomplished by standard procedures. In certain embodiments, the cell-containing sample is a whole blood cell lysate. Kahn et al. (Biochem. Biophys. Res. Commun., 214:957-962 (1995)) and Tsubuki et al. (FEBS Lett., 344:229-233 (1994)) disclose that red blood cells contain endogenous proteinaceous inhibitors of the proteasome. However, endogenous proteasomal peptidase inhibitors can be inactivated in the presence of SDS at a concentration of about 0.05%. allowing red blood cell lysates and whole blood cell lysates to be assayed reliably. At this concentration of SDS, most if not all proteasomal peptidase activity is due to the 20S proteasome. Although purified 20S proteasome exhibits poor stability at 0.05% SDS, 20S proteasomal peptidase activity in cell lysates is stable under these conditions (Vaddi et al., U.S. Pat. No. 6,613,541).

In other embodiments, the cell-containing sample is a white blood cell lysate. Methods for obtaining white blood cells from blood are known in the art (Rickwood et al., Anal. Biochem., 123:23-31 (1982); Fotino et al., Ann. Clin. Lab. Sci., 1:131 (1971)). Commercial products useful for cell separation include without limitation Ficoll-Paque (Pharmacia Biotech) and NycoPrep (Nycomed). In some situations, white blood cell lysates provide better reproducibility of data than do whole blood cell lysates.

Variability in sample preparation of cell-containing samples can be corrected by normalizing the data by, for example, protein content or cell number. In certain embodiments, proteasomal peptidase activity in the sample may be normalized relative to the total protein content or proteasomal protein content in the sample (specific activity method). Total protein content in the sample can be determined using standard procedures, including, without limitation, Bradford assay and the Lowry method. In other embodiments, proteasomal peptidase activity in the sample may be normalized relative to cell number.

### Measuring Proteasome Level

In one embodiment, the quantity or concentration proteasomes may be measured by determining the amount of one or more proteasomal proteins in a sample. The polypeptides in the proteasome can be detected by an antibody which is detectably labeled, or which can be subsequently labeled. A variety of formats can be employed to determine whether a sample contains a proteasomal protein or proteins that bind to a given antibody. Immunoassay methods useful in the detection of proteasomal proteins include, but are not limited to, e.g., dot blotting, western blotting, protein chips, immunoprecipitation (IP), competitive and non-competitive protein binding assays, enzyme-linked immunosorbent assays (ELISA), and others commonly used and widely-described in scientific and patent literature, and many employed commercially.

Proteins from samples can be isolated using techniques that are well-known to those of skill in the art. The protein isolation methods employed can, e.g., be including, but not limited to, e.g., those described in Harlow & Lane, Antibodies: A Laboratory Manual (Cold Spring Harbor Laboratory Press, Cold Spring Harbor. N.Y. 1988). In some embodiments, proteasomal protein is extracted from the acellular body fluid sample. Plasma purification methods are known in the art such. See e.g., Cohn, E. J., et al., Am. Chem. Soc., 62:3396-3400.(1940); Cohn. E. J., et al., J. Am. Chem. Soc., 72:465-474 (1950); Pennell, R. B., Fractionation and isolation of purified components by precipitation methods, pp. 9-50. In The Plasma Proteins, Vol. 1, F. W. Putman (ed.). Academic Press, New York (1960); and U.S. Pat. No. 5,817,765.

Antibodies can be used in methods, including, but not limited to, e.g., western blots or ELISA, to detect the expressed protein complexes. In such uses, it is possible to immobilize either the antibody or proteins on a solid support. Supports or carriers include any support capable of binding an antigen or an antibody. Well-known supports or carriers include, but are not limited to, e.g., glass, polystyrene, polypropylene, polyethylene, dextran, nylon, amylases, natural and modified celluloses, polyacrylamides, gabbros, and magnetite.

Antibodies may be specific for one or more proteins that comprise the proteasomal complex. In one embodiment, the quantity or concentration of proteasomes in a sample is determined by detecting the quantity or concentration of one or more proteins that interact to form the proteasomal complex. In one embodiment, the quantity or concentration of proteasomes in a sample is determined using a polyclonal antibody to the 20S Proteasome core subunits. In other embodiments, the quantity or concentration of proteasomes in a sample is determined using a polyclonal or a monoclonal antibody directed to one or more proteins including, but not limited to, Ki-67 protein, 19S Regulator ATPase Subunit Rpt4, 19S Proteasome S5A-Subunit; 19S Proteasome S5A-Subunit,; 19S Proteasome, S6-Subunit; 20S Proteasome α1, 2, 3, 5, 6, & 7-Subunits; 20S Proteasome α1-Subunit; 20S Proteasome α3-Subunit; 20S Proteasome α5-Subunit; 20S Proteasome α7-Subunit; 20S Proteasome β1-Subunit; 20S Proteasome β3-Subunit; 20S Proteasome β4-Subunit; 20S Proteasome β5i-Subunit; 26S Proteasome S4-Subunit; 26S Proteasome, S7-Subunit; Proteasome Activator PA700 Subunit 10B; 19S Regulator ATPase Subunit Rpt1; and 19S Regulator non-ATPase Subunit Rpn10.

Methods of generating antibodies are well known in the art, see, e.g., Sambrook, et al., 1989, Molecular Cloning: A Laboratory Manual, Second Edition, Cold Spring Harbor Press, Plainview, N.Y. Antibodies may be detectably labeled by methods known in the art. Labels include, but are not limited to, radioisotopes such as ³H, ¹⁴C, ³⁵S, ³²P, ¹²³I, ¹²⁵I, ¹³¹I), enzymes (e.g., peroxidase, alkaline phosphatase, beta-galactosidase, luciferase, alkaline phosphatase, acetylcholinesterase and glucose oxidase), enzyme substrates, luminescent substances (e.g., luminol), fluorescent substances (e.g., FITC, rhodamine, lanthanide phosphors), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags) and colored substances. In binding these labeling agents to the antibody, the maleimide method (Kitagawa, T., et al., J. Biochem., 79:233-236 (1976)), the activated biotin method (Hofmann, K., et al., J. Am. Chem. Soc., 100:3585 (1978)) or the hydrophobic bond method, for instance, can be used.

In some embodiments, labels are attached via spacer arms of various lengths to reduce potential steric hindrance. Antibodies may also be coupled to electron dense substances, such as ferritin or colloidal gold, which are readily visualized by electron microscopy.

Where a radioactive label is used as a detectable substance, proteins may be localized by autoradiography. The results of autoradiography may be quantitated by determining the density of particles in the autoradiographs by various optical methods, or by counting the grains.

The antibody or sample may be immobilized on a carrier or solid support which is capable of immobilizing cells, antibodies, etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against one or more proteins that comprise a proteasome. Antibodies to proteasomal proteins are available commercially through multiple sources. For example, polyclonal antibodies directed to proteasome core subunit are available from Biomol International, Cat. No. PW8155-0100 (Plymouth, Pa.). Monoclonal antibodies directed to proteasome α subunit are available from Biomol International, Cat. No. PW8100 (Plymouth, Pa.).

Immunoassays, or assays to detect an antigen using an antibody, are well known in the art and can take many forms, e.g., radioimmunoassay, immunoprecipitation, Western blotting, enzyme-linked immunosorbent assay (ELISA), and 2-site or sandwich immunoassay.

In one embodiment, a sandwich ELISA is used. In this assay, two antibodies to different segments, or epitopes, of the antigen are used. The first antibody (capture antibody) is coupled to a solid support. When a sample of bodily fluid is contacted with the capture antibody on the solid support, the antigen contained in the bodily fluid is captured on the solid support through a specific interaction between antigen and antibody, resulting in the formation of a complex. Washing of the solid support removes unbound or non-specifically hound antigen. Subsequent exposure of the solid support to a detectably-labeled second antibody (detection antibody) to the antigen (generally to a different epitope than the capture antibody) enables the detection of bound or captured antigen. As would he readily recognized by one of skill in the art, assaying additional markers in parallel to assaying for proteasomal protein is possible with the use of distinct pairs of specific antibodies, each of which is directed against a different marker.

In an illustrative embodiment, a electro-chemiluminescent sandwich immunoassay is used. In this assay, two antibodies to different segments, or epitopes, of the antigen are used. For instance, antibody to one or more proteasomal proteins is coated on plates to capture the proteasomes. The antibody may be a mouse monoclonal antibody to proteasome alpha subunit. A sample is contacted to the plate, and after incubation under appropriate binding conditions, the plate is washed. After the wash, primary detection antibody, which binds to the one or more proteasomal proteins, is added to each well and incubated. After another wash, a Sulfo-tag labeled secondary antibody (capable of binding to the primary antibody) is added to each well and incubated for another hour. After a final wash, a MSD read buffer is added and signal is detected by MSD Sector2400 (MSD, Gaithersburg, Md.).

Relative or actual amounts of proteasomes in body fluids can be determined by methods well known in the art. See. e.g., Drach, J., et al., Cytometry. 10(6):743-749 (1989). For example, a standard curve can be obtained in the ELISA using known amounts of proteasomes, i.e., proteasome standards. The actual amount of the proteasomes in a body fluid may thus be determined using the standard curve. Another approach that does not use a standard curve is to determine the dilution of body fluid that gives a specified amount of signal. The dilution at which 50% of the signal is obtained is often used for this purpose. In this case, the dilution at 50% maximal binding of proteasomes in a patient body fluid is compared with the dilution at 50% of maximal binding for proteasomes obtained in the same assay using a reference sample (i.e., a sample taken from the corresponding bodily fluid of normal individuals, free of proliferative disorders).

Monoclonal or polyclonal antibodies may be used as the capture and detection antibodies in sandwich immunoassay systems. Monoclonal antibodies are specific for single epitope of an antigen and allow for detection and quantitation of small differences in antigen. Polyclonal antibodies can be used as the capture antibody to capture large amounts of antigen or can be used as the detection antibody. A monoclonal antibody can be used as the either the capture antibody or the detection antibody in the sandwich assay to provide greater specificity. In some embodiments, polyclonal antibodies are used as the capture antibody and monoclonal antibodies are used as the detection antibody.

One consideration in designing a sandwich ELISA is that the capture and detection antibodies should be generated against or recognize “non-overlapping” epitopes. The phrase “non-overlapping” refers to epitopes, which are segments or regions of an antigen that are recognized by an antibody, that are sufficiently separated from each other such that an antibody for each epitope can bind simultaneously. That is, the binding of one antibody (e.g., the capture antibody) to a first epitope of the antigen should not interfere with the binding of a second antibody (e.g., the detection antibody) to a second epitope of the same antigen. Capture and detection antibodies that do not interfere with one another and can bind simultaneously are suitable for use in a sandwich ELISA.

Methods for immobilizing capture antibodies on a variety of solid surfaces are well-known in the art. The solid surface may be composed of any of a variety of materials, for example, glass, quartz, silica, paper, plastic, nitrocellulose, nylon, polypropylene, polystyrene, or other polymers. The solid support may be in the form of beads, microparticles, microspheres, plates which are flat or comprise wells, shallow depressions, or grooves, microwell surfaces, slides, chromatography columns, membranes, filters, or microchips. In one embodiment, the solid support is a microwell plate in which each well comprises a distinct capture antibody to a specific marker so that multiple markers may be assayed on a single plate. In another embodiment, the solid support is in the form of a bead or microparticle. These beads may be composed of, for example, polystyrene or latex. Beads may be of a similar size or may be of varying size. Beads may be approximately 0.1 μm-10 μm in diameter or may be as large as 50 μm-100 μm in diameter.

Methods of identifying the binding of a specific binding agent to proteasomes are known in the art and vary dependent on the nature of the label. In suitable embodiments, the detectable label is a fluorescent dye. Fluorescent dyes are detected through exposure of the label to a photon of energy of one wavelength, supplied by an external source such as an incandescent lamp or laser, causing the fluorophore to be transformed into an excited state. The fluorophore then emits the absorbed energy in a longer wavelength than the excitation wavelength which can be measured as fluorescence by standard instruments containing fluorescence detectors. Exemplary fluorescence instruments include spectrofluorometers and microplate readers, fluorescence microscopes, fluorescence scanners, and flow cytometers.

In one embodiment, a sandwich assay is constructed in which the capture antibody is coupled to a solid support such as a bead or microparticle. Captured antibody-antigen complexes, subsequently hound to detection antibody, are detected using flow cytometry and is well-known in the art. Flow cytometers hydrodynamically focus a liquid suspension of particles (e.g., cells or synthetic microparticles or beads) into an essentially single-file stream of Particles such that each particle can be analyzed individually. Flow cytometers are capable of measuring forward and side light scattering which correlates with the size of the particle. Thus, particles of differing sizes or fluorescent characteristics may be used in invention methods simultaneously to detect distinct markers. Fluorescence at one or more wavelengths can be measured simultaneously. Consequently, particles can be sorted by size and the fluorescence of one or more fluorescent labels can be analyzed for each particle. Exemplary flow cytometers include the Becton-Dickinson Immunocytometry Systems FACSCAN. Equivalent flow cytometers can also he used in the invention methods.

### Measuring Proteasome Activity

Proteasome activity in the test sample can be measured by any assay method suitable for determining 20S or 26S proteasome peptidase activity. (See. e.g., Vaddi et al., U.S. Pat. No. 6,613,541; McCormack et al., Biochemistry, 37:7792-7800 (1998)); Driscoll and Goldberg, J. Biol. Chem., 265:4789 (1990); Orlowski et al., Biochemistry,32:1563 (1993)). In a suitable embodiment, a substrate having a detectable label is provided to the reaction mixture and proteolytic cleavage of the substrate is monitored by following disappearance of the substrate or appearance of a cleavage product. Detection of the label may be achieved, for example, by, fluorometric, colorimetric, or radiometric assay.

Substrates for use in determining proteasomal peptidase activity may be chosen based on the selectivity of each peptidase activity. For example, the chymotrypsin-like peptidase preferentially cleaves peptides on the carboxyl side of tyrosine, tryptophan, phenylalanine, leucine, and methionine residues. The trypsin-like peptidase preferentially cleaves peptides on the carboxyl side of arainine and lysine residues. The caspase-like peptidase (or peptidylglutamyl-peptide hydrolase) preferentially cleaves peptides at glutamic acid and aspartic acid residues. Based on these selectivities, the skilled artisan can choose a specific substrates for each peptidase.

Suitable substrates for determining 26S proteasome activity include, without limitation lysozyme, α-lactalbumin, β-lactoglobulin, insulin b-chain, and ornithine decarboxylase. When 26S proteasome activity is to he measured, the substrate is typically ubiquitinated or the reaction mixture further contains ubiquitin and uhiquitination enzymes.

In some embodiments, the substrate is a peptide less than 10 amino acids in length. In one embodiment, the peptide substrate contains a cleavable fluorescent label and release of the label is monitored by fluorometric assay. Non-limiting examples of substrates to measure trypsin-like activity include N-(N-benzoylvalylglycylarginyl)-7-amino-4-methylcoumarin (Bz-Val-Gly-Arg-AMC), N-(N-carbobenzyloxycarbonylleucylleucylarginyl)-7-amino-4-methylcoumarin (Z-Leu-Leu-Arg-AMC), Ac-Arg-Leu-Arg-AMC, and Boc-Leu-Arg-Arg-AMC. Non-limiting examples of substrates to measure caspase-like activity include N-(N-carbobenzyloxycarbonylleucylleucylglutamyl)-2-naphthylamine (Z-Leu-Leu-Glu-2NA), N-(N-carbobenzyloxycarbonylleucylleucylglutamyl)-7-amino-4-methylcoumarin (Z-Leu-Leu-Glu-AMC), and acetyl-L-glycyl-L-prolyl-L-leucyl-L-aspartyl-methylcoumarin (Ac-Gly-Pro-Leu-Asp-AMC). Non-limiting examples of substrates to measure chymotrypsin-like activity include N-(N-suceinylleucylleucylvalyltyrosyl)-7-amino-4-methylcournarin (Suc-Leu-Leu-Val-Tyr-AMC), Z-Gly-Gly-Leu-2NA, Z-Gly-Gly-Leu-AMC, and Suc-Arg-Pro-Phe-His-Leu-Leu-Val-Tyr-AMC.

Suitable substrates for measuring the chymotrypsin-like, caspase-like, and trypsin-like activities of the proteasome are Suc-Leu-Leu-Val-Tyr-AMC, Z-Leu-Leu-Glu-AMC, and Bz-Val-Gly-Arg-AMC, respectively, and the release of the cleavage product, AMC, can be monitored at 440 nm (λₑₓ=380 nm). Cleavage due to a particular peptidase may be determined by, for example, using a substrate specific for that peptidase and assaying that activity independent of other peptidases.

In certain embodiments, the reaction mixture further contains a 20S proteasome activator. Activators include those taught in Coux et al. (Ann. Rev. Biochem., 65:801-847 (1995)), such as PA28 or sodium dodecyl sulfate (SDS). However, SDS is not compatible with Bz-Val-Gly-Arg-AMC, therefore when Bz-Val-Gly-Arg-AMC is chosen as the substrate. PA28 is used instead of SDS to activate the proteasome.

### Diagnosis and Staging of Liver Disease States

Provided herein are methods of diagnosing and staging chronic liver disease (CLD) and advanced liver fibrosis. In certain embodiments, the level of one or more proteasomal peptidase activities in a test sample from a patient is used in the diagnosis of liver disease. In some embodiments, the specific activity level of one or more proteasomal peptidases (e.g., Ch-L/p, Tr-L/p, and Cas-L/p) in a test sample are used to diagnose a disease. In these embodiments, the level of proteasome activity measured in the test sample is normalized to the level of one or more proteasomal proteins to provide a specific activity value for the one or more proteasomal peptidases. The specific activity value may be compared to a reference value to determine if the levels of specific activity are elevated or reduced relative to the reference value. Typically, the reference value is the specific activity measured in a comparable sample from one or more healthy individuals. An increase or decrease in the specific activity may be used in conjunction with clinical factors other than proteasomal peptidase activity to diagnose a liver disease.

Association between a pathological state (e.g., a liver disease) and the aberration of a specific activity level of one or more proteasomal peptidases can be readily determined by comparative analysis in a normal population and an abnormal or affected population. Thus, for example, one can study the specific activity level of one or more proteasomal peptidases in both a normal population and a population affected with a particular pathological state. The study results can be compared and analyzed by statistical means. Any detected statistically significant difference in the two populations would indicate an association. For example, if the specific activity is statistically significantly higher in the affected population than in the normal population, then it can be reasonably concluded that higher specific activity is associated with the pathological state.

Statistical methods can be used to set thresholds for determining when the specific activity level in a subject can be considered to be different than or similar to a reference level. In addition, statistics can be used to determine the validity of the difference or similarity observed between a patient's specific activity level and the reference level. Useful statistical analysis methods are described in L. D. Fisher & G. vanBelle, Biostatistics: A Methodology for the Health Sciences (Wiley-Interscience, NY, 1993). For instance, confidence (“p”) values can be calculated using an unpaired 2-tailed t test, with a difference between groups deemed significant if the p value is less than or equal to 0.05. As used herein a “confidence interval” or “CI” refers to a measure of the precision of an estimated or calculated value. The interval represents the range of values, consistent with the data that is believed to encompass the “true” value with high probability (usually 95%). The confidence interval is expressed in the same units as the estimate or calculated value. Wider intervals indicate lower precision; narrow intervals indicate greater precision. Suitable confidence intervals of the invention are 90%, 95%, 97.5%, 98%, 99%, 99.5%, 99.9% and 99.99%. A “p-value” as used herein refers to a measure of probability that a difference between groups happened by chance. For example, a difference between two groups having a p-value of 0.01 (or p=0.01) means that there is a 1 in 100 chance the result occurred by chance. Suitable p values are 0.1, 0.05, 0.025, 0.02, 0.01, 0.005, 0.001, and 0.0001. Confidence intervals and p-values can be determined by methods well-known in the art. See, e.g., Dowdy and Wearden, Statistics for Research, John Wiley & Sons, New York, 1983. Exemplary statistical tests for associating a prognostic indicator with a predisposition to an adverse outcome are described hereinafter.

Once an association is established between a specific activity and a pathological state, then the particular physiological state can be diagnosed or detected by determining whether a patient has the particular aberration, i.e. elevated or reduced specific activity levels.

The term “elevated levels” or “higher levels” as used herein refers to levels of a specific activity that are higher than what would normally he observed in a comparable sample from control or normal subjects (i.e., a reference value). In some embodiments, “control levels” (i.e., normal levels) refer to a range of specific activity levels that would be normally he expected to be observed in a mammal that does not have a liver disease. A control level may be used as a reference level for comparative purposes. “Elevated levels” refer to specific activity levels that are above the range of control levels. The ranges accepted as “elevated levels” or “control levels” are dependent on a number of factors. For example, one laboratory may routinely determine the specific activity of an enzyme in a sample that are different than the specific activity obtained for the same sample by another laboratory. Also, different assay methods may achieve different value ranges. Value ranges may also differ in various sample types, for example, different body fluids or by different treatments of the sample. One of ordinary skill in the art is capable of considering the relevant factors and establishing appropriate reference ranges for “control values” and “elevated values” of the present invention. For example, a series of samples from control subjects and subjects diagnosed with liver disease can be used to establish ranges that are “normal” or “control” levels and ranges that are “elevated” or “higher” than the control range.

Similarly, “reduced levels” or “lower levels” as used herein refer to levels of a peptidase specific activity that are lower than what would normally he observed in a comparable sample from control or normal subjects (i.e., a reference value). In some embodiments, “control levels” (i.e. normal levels) refer to a range of specific activity levels that would be normally be expected to be observed in a mammal that does not have a liver disease and “reduced levels” refer to proteasome activity levels that are below the range of such control levels.

The specific activity level of one or more peptidases in a test sample can be used in conjunction with clinical factors other than specific activity to diagnose a disease. Clinical factors of particular relevance in the diagnosis of liver disease include, but are not limited to, the patient's medical history, a physical examination of the patient, and liver enzymes. In some embodiments, the specific activity level at one or more proteasomal peptides is combined with one or more additional liver disease markers to improve diagnostic sensitivity and specificity. Exemplary liver disease markers include, but are not limited to ALT, AST, ALP, bilirubin, and serum albumin.

Alanine transaminase (ALT), also called Serum Glutamic Pyruvate Transaminase (SGPT) or Alanine aminotransferase (ALAT), is an enzyme present in hepatocytes (liver cells). When a cell is damaged, it leaks this enzyme into the blood, where it is measured. ALT rises dramatically in acute liver damage, such as viral hepatitis or paracetamol (acetaminophen) overdose. Elevations are often measured in multiples of the upper limit of normal (ULN).

Aspartate transaminase (AST), also called Serum Glutamic Oxaloacetic Transaminase (SGOT) or aspartate aminotransferase (ASAT), is similar to ALT in that it is another enzyme associated with liver parenchymal cells. It is raised in acute liver damage, but is also present in red blood cells, and cardiac and skeletal muscle and is therefore not specific to the liver. The ratio of AST to ALT is sometimes useful in differentiating between causes of liver damage.

Alkaline phosphatase (ALP) is an enzyme in the cells lining the biliary ducts of the liver. ALP levels in plasma will rise with large bile duct obstruction, intrahepatic cholestasis or infiltrative diseases of the liver.

Bilirubin is a breakdown product of heme (a part of haemoglobin in red blood cells). The liver is responsible for clearing the blood of bilirubin. It does this by the following mechanism: bilirubin is taken up into hepatocytes, conjugated (modified to make it water-soluble), and secreted into the bile, which is excreted into the intestine.

### UPS Signature Models

In some embodiments, a “UPS signature model” is used for the diagnosis and staging of liver disease, including, but not limited to chronic liver disease and advanced liver fibrosis. The model may include UPS components, proteasome and ubiquitin, proteasome enzymatic activities, Ch-L, Cas-L, Tr-L, Ch-L/P, Cas-L/P, and Tr-L/P, with gender and age, alone and in combination with conventional liver disease markers. ALT, ALP, AST, bilirubin, and serum albumin. In illustrative embodiments, the UPS signature model yields excellent diagnostic characteristics with a sensitivity of 96.4% and a specificity of 98.5%. Rather than using cutoffs from individual marker, the UPS signature model statistically weights each marker and uses the cumulative probabilities of the response categories rather than individual probability.

In one embodiment, the diagnosis of chronic liver disease is accomplished by obtaining a sample of serum from the subject and determining the level of Tr-L/p, ubiquitin, ALT, ASP, and ALP. In this embodiment, intermediate value (y) is calculated as follow's:

y=exp [−X+(C₁×Age)+(C₂×Tr-L/p)+(C₃×Ubiquitin)+(C₄×ALT)+(C₅×ASP)+(C₆×ALP)]

wherein X is from −9.706 to −2.7958 inclusive; C₁ is from 0.0957 to 0.1835 inclusive; C₂ is from −0.1300 to −0.0578 inclusive; C₃ is from −0.0381 to −0.0143 inclusive; C₄ is from −0.1827 to −0.0965 inclusive; C₅ is from 0.2165 to 0.4107 inclusive; C₆ is from −0.0508 to −0.0222 inclusive; and wherein, age is provided in years; normalized Tr-L (Tr-L/p) is reported in pmol product /sec/pg, proteasome; ubiquitin is reported in mg/dL; ASP, ALT, and ALP are reported in IU/L.

In a particular embodiment, the intermediate value (y) is calculated as follows:

y=exp [6.2540+(0.1396×Age)+(−0.0939×Tr-L/p)+(−0.0262×Ubiquitin)+(−0.1396×ALT)+(0.3136×ASP)+(−0.0365×ALP)]

wherein, age is provided in years; normalized Tr-L (Tr-L/p) is reported in pmol product /sec/pg proteasome; ubiquitin is reported in mg/dL; ASP, ALT, and ALP are reported in IU/L.

The intermediate value (y) is input into a second equation to determine the end value or UPS score, wherein

UPS Score=y/(1+y).

A UPS Score greater than or equal to a reference score of about 0.5 is predictive of CLD in a subject. A UPS Score less than a reference score of about 0.5 is predictive of the absence of CLD in the subject. In certain embodiments, this reference score may be from 0.25 to 0.75 inclusive, or from 0.4 to 0.6 inclusive, or from 0.45 to 0.55 inclusive. Alternatively, this reference score may be 0.4, 0.5, or 0.6. The above numbers are subject to 5% variation.

In one embodiment, the diagnosis of advanced liver fibrosis is accomplished by obtaining a sample of serum from the subject and determining the level of Tr-L/p, ALT, bilirubin, and albumin. In this embodiment, intermediate value (y) is calculated as follows:

y=exp [−X+(C₁×Age)+(C₂×Tr-L/p)+(C₃×ALT)+(C₄×+(C₅×Albumin)]

wherein X is from 0.7274 to 16.3490 inclusive; C₁ is from 0.0368 to 0.1682 inclusive; C₂ is from 0.0494 to 0.1556 inclusive; C₃ is from 0.0173 to 0.0433 inclusive; C₄ is from 0.7512 to 3.9666 inclusive; and C₅ is from −6.4955 to −2.6163 inclusive; wherein, age is provided in years; normalized Tr-L (Tr-L/p) is reported in pmol product /sec/pg proteasome; ALT is reported in IU/L; and bilirubin and ubiquitin are reported in mg/dL.

In a particular embodiment, the intermediate value (y) is calculated as follows:

y=exp [−8.5382+(0.1025×Age)+(0.1025×Tr-L/p)+(0.0303×ALT)−(2.3589×Bilirubin)+(−4.5559×Albumin)]

wherein age is provided in years; normalized Tr-L (Tr-L/p) is reported in pmol product /sec/pg proteasome; ALT is reported in IU/L; and bilirubin and ubiquitin are reported in mg/dL.

The intermediate value (y) is input into a second equation to determine the end value or UPS score, wherein

UPS Score=y/(1+y).

A UPS Score greater than or equal to a reference score of about 0.5 is predictive of advanced liver fibrosis in a subject, i.e., a Metavir score≧F3. A UPS Score less than a reference score of about 0.5 is predictive of the absence of advanced liver fibrosis in the subject, i.e., a Metavir score≦F2. In certain embodiments, this reference score may be from 0.25 to 0.75 inclusive, or from 0.4 to 0.6 inclusive, or from 0.45 to 0.55 inclusive. Alternatively, this reference score may be 0.4, 0.5, or 0.6. The above numbers are subject to 5% variation.

One of skill in the art would recognize that the concentrations or activities of the markers could be provided in unit's other than the ones recited above. In this case, one would generate an equivalent equation to determine the intermediate value by converting the units as recited above to other units using a mathematical function. The inverse of that function would be performed on the coefficient of that marker.

In another aspect, the invention provides a system for diagnosing the presence of liver disease in an individual. The system comprises an input device in data communication with a processor, which is in data communication with an output device.

The input device is used for entry of data including levels of Ch-L/p, Cas-L/p, Tr-L/p, Ch-L, Tr-L, Cas-L, ALT, ALP, AST, ubiquitin, bilirubin, and serum albumin as determined from a sample from the individual, and data for age and gender. Data may be entered manually by an operator of the system using a keyboard or keypad. Alternatively, data may be entered electronically, when the input device is a cable, in data communication with a computer, a network, a server, or analytical instrument.

The processor comprises software for computing a UPS Score, and using the end value to diagnose chronic liver disease. The processor computes the UPS Score using an algorithm, wherein the algorithm is UPS Score=y/(1+y), wherein (y) is calculated as follows:

y=exp [−X(C₁+Age)+(C₂×Tr-L/p)+(C₃×Ubiquitin)+(C₄×ALT)+(C₅×ASP)+(C₆×ALP)]

wherein X is from −9.706 to −2.7958 inclusive; C₁ is from 0.0957 to 0.1835 inclusive; C₂ is from −0.1300 to −0.0578 inclusive: C₃ is from −0.0381 to −0.0143 inclusive; C₄ is from −0.1827 to −0.0965 inclusive; C₅ is from 0.2165 to 0.4107 inclusive; C₆ is from −0.0508 to −0.0222 inclusive; and wherein, age is provided in years; normalized Tr-L (Tr-L/p) is reported in pmol product /sec/pg proteasome; ubiquitin is reported in mg/dL, ASP, ALT, and ALP are reported in IU/L.

The processor further compares the UPS score to a reference score to diagnose the presence of chronic liver disease, wherein a UPS score greater than or equal to a reference score of 0.5 is predictive of chronic liver disease. A UPS score less than a reference score of about 0.5 is predictive of an absence of chronic liver disease. In certain embodiments, this reference score may be from 0.25 to 0.75 inclusive, or from 0.4 to 0.6 inclusive, or from 0.45 to 0.55 inclusive. Alternatively, this reference score may be 0.4, 0.5, or 0.6. The above numbers are subject to 5% variation.

The processor comprises software for computing a UPS Score, and using the end value to stage CLD, i.e., to determine the presence of absence of advanced liver fibrosis. The processor computes the UPS Score using an algorithm, wherein the algorithm is UPS Score=y/(1+y), wherein (y) is calculated as follows:

y=exp [−X+(C₁×Age)+(C₂×Tr-L/p)+(C₃×ALT)+(C₄×Bilirubin)+(C₅×Albumin)]

wherein X is from 0.7274 to 16.3490 inclusive; C₁ is from 0.0368 to 0.1682 inclusive; C₂ is from 0.0494 to 0.1556 inclusive; C₃ is from 0.0173 to 0.0433 inclusive; is from 0.7512 to 3.9666 inclusive; and C₅ is from −6.4955 to −2.6163 inclusive; wherein, age is provided in years; normalized Tr-L (Tr-L/p) is reported in pmol product /sec/pg proteasome; ALT is reported in IU/L; and bilirubin and ubiquitin are reported in mg/dL.

The processor further compares the UPS score to a reference score to diagnose the presence of advanced liver fibrosis, wherein a UPS score greater than or equal to a reference score of 0.5 is predictive of advanced liver fibrosis, i.e., a Metavir score≧F3. A UPS score less than a reference score of about 0.5 is predictive of an absence of advanced liver fibrosis, i.e., a Metavir score≦F2. In certain embodiments, this reference score may be from 0.25 to 0.75 inclusive, or from 0.4 to 0.6 inclusive, or from 0.45 to 0.55 inclusive. Alternatively, this reference score may be 0.4, 0.5, or 0.6. The above numbers are subject to 5% variation.

The data output device, in data communication with the processor, receives the diagnosis from the processor and provides the diagnosis to the system operator. The output device can consist of, for example, a video display monitor or a printer.

Monitoring Progression and/or Treatment

In one aspect, the specific activity level of one or more proteasomal peptidases (e.g., Ch-L/p, Tr-L/p, and Cas-L/p) in a biological sample of a patient is used to monitor the effectiveness of treatment or the prognosis of disease. In some embodiments, the specific activity level of one or more proteasomal peptidases in a test sample obtained from a treated patient can be compared to the level from a reference sample obtained from that patient prior to initiation of a treatment. Clinical monitoring of treatment typically entails that each patient serve as his or her own baseline control. In some embodiments, test samples are obtained at multiple time points following administration of the treatment. In these embodiments, measurement of specific activity level of one or more proteasomal peptidases in the test samples provides an indication of the extent and duration of in vivo effect of the treatment.

### Determining Prognosis

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

Additionally, a change in a clinical factor from a baseline level may impact a patient's prognosis, and the degree of change in level of the clinical factor may be related to the severity of adverse events. Statistical significance is often determined by comparing two or more populations, and determining a confidence interval and/or a p value.

Multiple determinations of proteasomal specific activity levels can be made, and a temporal change in activity can be used to determine a prognosis. For example, comparative measurements are made of the specific activity of an acellular body fluid in a patient at multiple time points, and a comparison of a specific activity value at two or more time points may be indicative of a particular prognosis.

A prognosis is often determined by examining one or more clinical factors and/or symptoms that correlate to patient outcomes. As described herein, the specific activity level of a proteasomal peptidase is a clinical factor useful in determining prognosis. The skilled artisan will understand that associating a clinical factor with a predisposition to an adverse outcome may involve statistical analysis.

In certain embodiments, the levels of specific activity of one or more proteasomal peptidases are used as indicators of an unfavorable prognosis. According to the method, the determination of prognosis can be performed by comparing the measured specific activity level to levels determined in comparable samples from healthy individuals or to levels known to corresponding with favorable or unfavorable outcomes. The absolute specific activity levels obtained may depend on an number of factors, including, but not limited to, the laboratory performing the assays, the assay methods used, the type of body fluid sample used and the type of disease a patient is afflicted with. According to the method, values can be collected from a series of patients with a particular disorder to determine appropriate reference ranges of specific activity for that disorder. One of ordinary skill in the art is capable of performing a retrospective study that compares the determined specific activity levels to the observed outcome of the patients and establishing ranges of levels that can be used to designate the prognosis of the patients with a particular disorder. For example, specific activity levels in the lowest range would be indicative of a more favorable prognosis, while specific activity levels in the highest ranges would be indicative of an unfavorable prognosis. Thus, in this aspect the term “elevated levels” refers to levels of specific activity that are above the range of the reference value. In some embodiments patients with “high” or elevated” specific activity levels have levels that are higher than the median activity in a population of patients with that disease. In certain embodiments, “high” or “elevated” specific activity levels for a patient with a particular disease refers to levels that are above the median values for patients with that disorder and are in the upper 40% of patients with the disorder, or to levels that are in the upper 20% of patients with the disorder, or to levels that are in the upper 10% of patients with the disorder, or to levels that are in the upper 5% of patients with the disorder.

Because the level of specific activity in a test sample from a patient relates to the prognosis of a patient in a continuous fashion, the determination of prognosis can be performed using statistical analyses to relate the determined specific activity levels to the prognosis of the patient. A skilled artisan is capable of designing appropriate statistical methods. For example, the methods may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses.

In certain embodiments, the prognosis or patients with liver disease can be correlated to the clinical outcome of the disease using the specific activity level and other clinical factors. Simple algorithms have been described and are readily adapted to this end. The approach by Giles et al., British Journal of Hematology, 121:578-585, is exemplary. As in Giles et al., associations between categorical variables (e.g., proteasome activity levels and clinical characteristics) can be assessed via crosstabulation and Fisher's exact test. Unadjusted survival probabilities can be estimated using the method of Kaplan and Meier. The Cox proportional hazards regression model also can be used to assess the ability of patient characteristics (such as proteasome activity levels) to predict survival, with ‘goodness of fit’ assessed by the Grambsch-Therneau test, Schoenfeld residual plots, martingale residual plots and likelihood ratio statistics (see Grambsch et al, 1995). In some embodiments, this approach can be adapted as a simple computer program that can be used with personal computers or personal digital assistants (PDA). The prediction of patients' survival time in based on their proteasome activity levels can be performed via the use of a visual basic for applications (VBA) computer program developed within Microsoft® Excel. The core construction and analysis may be based on the Cox proportional hazard models. The VBA application can be developed by obtaining a base hazard rate and parameter estimates. These statistical analyses can be performed using a statistical program such as the SAS® proportional hazards regression, PHREG, procedure. Estimates can then be used to obtain probabilities of surviving from one to 24 months given the patient's covariates. The program can make use of estimated probabilities to create a graphical representation of a given patient's predicted survival curve. In certain embodiments, the program also provides 6-month, 1-year and 18-month survival probabilities. A graphical interface can be used to input patient characteristics in a user-friendly manner.

In some embodiments, multiple prognostic factors, including specific activity level, are considered when determining the prognosis of a patient. For example, the prognosis of a liver disease patient may be determined based on specific activity and one or more prognostic factors selected from the group consisting of activity of liver enzymes, bilirubin, serum albumin, performance status, age, gender and previous diagnosis. In certain embodiments, other prognostic factors may be combined with the specific activity level or other biomarkers in the algorithm to determine prognosis with greater accuracy.

### Kits

A kit may be used for conducting the diagnostic and prognostic methods described herein. Typically, the kit should contain, in a carrier or compartmentalized container, reagents useful in any of the above-described embodiments of the diagnostic method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a proteasome. The antibodies may be labeled with a detectable marker such as radioactive isotopes, or enzymatic or fluorescence markers. Alternatively, secondary antibodies such as labeled anti-IgG and the like may be included for detection purposes. In addition, reagents to detect the activity of one or more proteasomal peptidases may be provided. Optionally, the kit can include standard proteasomes prepared or purified for comparison purposes. Instructions for using the kit or reagents contained therein are also included in the kit.

### EXAMPLES

The present methods and kits, thus generally described, will be understood more readily by reference to the following examples, which are provided by way of illustration and are not intended to be limiting of the present methods and kits.

In the example, the “UPS signature” profile in patients with CLD and a healthy control population was evaluated. The data demonstrate that the UPS signature profile, combined with other liver function tests, may be used in methods for diagnosis and staging of HCV-related CLD in patients. The following is a description of the materials and experimental procedures used in the example.

### Materials and Methods

Study Subjects. A total of 189 subjects were studied: 55 patients (65% men; median [range] age=55 [25-75] yr) with HCV-related CLD who showed no evidence of hepatocellular carcinoma (HCC) during ≧2 years of follow-up; HCV infection confirmed by polymerase chain reaction (PCR) analysis. CLD patient samples were obtained from the Liver Center, Harvard Medical School, Boston, Mass. 134 apparently healthy adults (36% men; median [range] age=35 [20-60] yr) with no known hepatitis or liver diseases, recruited at Quest Diagnostics Nichols Institute, San Juan Capistrano, Calif. All samples were collected with an IRB-approved protocol with informed consent. The sera were isolated from peripheral blood and stored at −80° C. until analysis.

Measurement of proteasome level. Proteasome levels were measured using an immunoassay based on electro-chemiluminescence technology (MesoSeale Discovery, Gaithersburg, Md.). A monoclonal antibody (MCP20, Biomol International, Cat. No. PW8100, Plymouth, Pa.) specific to proteasome alpha subunit was captured on a MSD goat anti-mouse plate. Proteasome standards (Biomol International, Cat. No. PW8720, Plymouth, Pa.), control and patient serum samples (1:20 dilution in MSD lyses buffer) were added to the wells and incubated at room temperature (RT) for 2 h. After washing, the detection antibody (Biomol International, Cat. No. PW8155-0100, Plymouth, Pa.), a rabbit polyclonal antibody against the proteasome core subunit, was added to the well and incubated at RT for 1 h. The plate was washed and incubated with sulfo-tag-labeled goat anti-rabbit antibody at RT for 1 h. Following the final wash, MSD read buffer was added to each well, and signal was detected on a MSD SECTOR™ Imager (MSD, Gaithersburg, Md.). The proteasome level in human serum (ng/mL) was calculated using proteasome standard curve. Sensitivity of the proteasome MSD assay was 100 pg/mL.

Measurement of circulating ubiquitin level. The level of ubiquitin in serum was detected by an immunoassay using electro-chemiluminescence-based technology. Briefly, a MSD plate was blocked with goat anti-mouse antibodies for 2 h. Then, an anti-ubiquitin monoclonal antibody (clone FK1, Cat. No. PW8805, Biomol International, Plymouth, Pa.) was coated on the MSD goat anti-mouse plate at 4° C. on a shaker for overnight. HeLa cell lysate was used for standards, and ubiquitin positive (Catalog No. 89899, Pierce, Rockford, Ill.) and negative controls were used in the assay. Serum samples were diluted 1:2 using the MSD lysis buffer. Controls, standards and serum samples were added to the wells and incubated at RT for 3 h on a shaker. During incubation, any ubiquitin present in samples or standards was specifically captured by the anti-ubiquitin. After washing, sulfo-tag-labeled anti-ubiquitin antibody was added to each well and incubated at RT for 1 h. After the final wash. MSD read buffer was added to the wells and signal was detected on an MSD SECTOR™ Imager (MSD, Gaithersburg, Md.). The ubiquitin levels (ng/mL) were extrapolated from reference standard curve. The sensitivity of the assay was 2 ng/mL.

Measurement of circulating proteasomal peptidase activities. The measurement of proteasome enzymatic activities has been previously described (Ma et al. Cancer. 2008, 112(6):1306-12). Briefly, chymotrypsin-like (Ch-L), caspase-like (Cas-L), and trypsin-like (Tr-L) activities were assayed by continuously monitoring the production of 7-amino-4-methylcoumarin (AMC) from fluorogenic peptides. The release of free AMC was measured on a SpectraMax Gemini EM instrument (Molecular Devices Corporation, Sunnyvale, Calif.) with the following parameters: excitation, 380 nm; emission, 460 nm; read interval, 1 min; read length, 30 min; temperature, 37° C. Enzymatic activities were quantitated by generating a standard curve of AMC (range, 0-8 μM). The slope of the AMC standard curve was then used as a conversion factor to calculate the activity of each individual sample as pmol AMC/second/mL serum. The specific activity of each proteasomal peptidase (Ch-L/p, Tr-L/p, and Cas-L/p) was also normalized to the amount of proteasomes in the sample and expressed as pmol AMC/sec/pg proteasome.

Determination of specific enzymatic activities of proteasomes. To determine the specific enzymatic activities of proteasomes, the level of the enzymatic activity was divided by the level of proteasome protein in the same quantity of serum sample. Therefore, three new values were generated: Ch-L specific activity (Ch-L/p)=Ch-L/proteasome Cas-L specific activity (Cas-L/p)=Cas-L/proteasome level; and Tr-L specific activity (Tr-L/p)=Tr-L/proteasome level.

Liver Function and Other Biochemical Tests. Levels of aspartate aminotransferase (AST), alanine aminotransferase (ALT), alkaline phosphatase (ALP), and bilirubin were determined by colorimetric methods on an Olympus AU640e (Olympus America Inc, Center Valley, Pa.). Albumin was measured with a fixed time nephelometric method on Dade Behring BNII-Nephelometry (Dade Behring Inc. Deerfield, Ill.)

### Results

Mode for Differentiating CLD from Normal Population. In an evaluation of UPS and liver function markers in CLD patients and healthy controls, relationships between CLD and 10 biochemical markers, gender, and age were investigated using univariate and multivariate logistic regression analysis. Multivariable models with different combinations of markers were constructed and compared using area under the receiver operating characteristic (AUROC) curve analysis. A single UPS-based model, yielding the most favorable AUROC with the fewest variables, was then selected (Table 1).

TABLE 1
Multivariate Logistic Regression Model for Differentiating Patients
with HCV-Related Chronic Liver Disease from Normal Population

| Variables | Coefficient | Coefficient SE | Coefficient P |
|---|---|---|---|
| Intercept | −6.2540 | 3.4582 | 0.0705 |
| Age | 0.1396 | 0.0439 | 0.0001 |
| Tr-L/p | −0.0939 | 0.0361 | 0.00003 |
| Ubiquitin | 0.0262 | 0.0119 | 0.0255 |
| ALT | −0.1396 | 0.0431 | <0.0001 |
| AST | 0.3136 | 0.0971 | <0.0001 |
| ALP | −0.0365 | 0.0143 | 0.0051 |

This model provided high accuracy for distinguishing CLD patients from healthy individuals, overall and when CLD patients were divided according to fibrosis score (Metavir score=0-1 or 2-4) (Table 2).

TABLE 2
Sensitivity and Specificity of UPS Signature Model for Differentiating
Patients with HCV-related Chronic Liver Disease (n = 55) from
Normal Population (n = 134)

| | Sensitivity | Specificity |
|---|---|---|
| Total patients with CLD | 90.9% | 98.5% |
| Metavir 0-1 (n = 26) | 84.5% | 98.5% |
| Metavir 2-4 (n = 28) | 96.4% | 98.5% |

A UPS signature cutoff score of >0.5 was considered to indicate CLD.

Model for Predicting Advanced Fibrosis. A second UPS signature model was created to differentiate CLD patients with Metavir 0-2 (n=24) from those with advanced fibrosis (Metavir 3-4, n=25) (Table 3). This model was highly predictive of advanced fibrosis (Table 4).

TABLE 3
Multivariate Logistic Regression Model for Predicting Advanced Fibrosis

| Variables | Coefficient | Coefficient SE | Coefficient P |
|---|---|---|---|
| Intercept | 8.5382 | 7.8108 | 0.2743 |
| Age | 0.1025 | 0.0657 | 0.1191 |
| Tr-L/p | 0.1025 | 0.0531 | 0.0537 |
| ALT | 0.0303 | 0.013 | 0.02 |
| Bilirubin | 2.3589 | 1.6077 | 0.1423 |
| Albumin | −4.5559 | 1.9396 | 0.0188 |

TABLE 4
Sensitivity and Specificity of UPS Signature Model for
Advanced Fibrosis in Patients with HCV-Rclated CLD

| | Sensitivity | Specificity |
|---|---|---|
| CLD with Metavir 3-4 (n = 25) | 88% | 91.7% |

These findings support the utility of models combining UPS and liver function markers for evaluating fibrosis in patients with CLD. As such, the use of ubiquitin-proteasome signature profile is useful in methods for diagnosing and staging patients with CLD.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably he practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for diagnosing chronic liver disease in a subject, the method comprising:
(a) determining the amount of one or more of chymotrypsin-like activity (Ch-L), trypsin-like activity (Tr-L), and caspase-like activity (Cas-L) in a sample from the subject;
(b) determining the amount of one or more of ALT, ASP, ALP, bilirubin, albumin, and ubiquitin in the sample;
(c) determining the amount of proteasomal protein in the sample and normalizing one or more of Ch-L, Tr-L, and Cas-L to calculate the specific activity;
(d) determining a single diagnostic score for the subject based on the results obtained in steps (b) and (c): and
(e) comparing the single diagnostic score to a reference score to determine a diagnosis of chronic liver disease in the subject.

2. The method of claim 1, wherein the amount of each of the Cas-L activity, Tr-L activity, and Ch-L activity are determined in a sample from the subject.

3. The method of claim 1, wherein the amount of at least one of ALT, ASP, and ALP are determined in a sample from the subject.

4. The method of claim 1, wherein the score is determined using the algorithm:
Score=y/(1+y)
wherein,
y=exp [−X+(C₁×Age)+(C₂×Tr-L/p)+(C₃×Ubiquitin)+(C₄×ALT)+(C₅×ASP)+(C₆×ALT)]
wherein X is from −9.706 to −2.7958 inclusive;
C₁ is from 0.0957 to 0.1835 inclusive;
C₂ is from −0.1300 to −0.0578 inclusive;
C₃ is from −0.0381 to −0.0143 inclusive;
C₄ is from −0.1827 to −0.0965 inclusive;
C₅ is from 0.2165 to 0.4107 inclusive;
C₆ is from −0.0508 to −0.0222 inclusive;
and wherein, age is in years; normalized Tr-L (Tr-L/p) is in pmol product /sec/pg proteasome; ubiquitin is in mg/dL; ASP; ALT, and ALP are in IU/L.

5. The method of claim 4, wherein
X is about −6.2540;
C₁ is about 0.1396;
C₂ is about −0.0939;
C₃ is about −0.062:
C₄ is about −0.1396:
C₅ is about 0,3136; and
C₆ is about −0.0365.

6. The method of claim 4, wherein the reference score is about 0.5 and a score less than about 0.5 is indicative of the absence of chronic liver disease in the subject.

7. The method of claim 4, wherein the reference score is about 0.5 and a score greater than or equal to about 0.5 is indicative of chronic liver disease in the subject.

8. The method of claim 1, wherein the sample is serum or plasma.

9. The method of claim 1, wherein the single diagnostic score is used for the choice of a suitable treatment for the subject.

10. A method for staging chronic liver disease and diagnosing advanced liver fibrosis in a subject, the method comprising:
(a) determining the amount of one or more of chymotrypsin-like activity (Ch-L), trypsin-like activity (Tr-L), and caspase-like activity (Cas-L) in a sample from the subject;
(b) determining the amount of one or more of ALT, ASP, ALP, bilirubin, albumin, and ubiquitin in the sample;
(c) determining the amount of proteasomal protein in the sample and normalizing one or more of Ch-L, Tr-L, and Cas-L to calculate the specific activity;
(d) determining a single diagnostic score for the subject based on the results obtained in steps (b) and (c); and
(c) comparing the single diagnostic score to a reference score that is predictive of a disease or symptom in order to determine the stage of chronic liver disease in the subject.

11. The method of claim 10, wherein the amount of each of the Cas-L activity, Tr-L activity, and Ch-L activity are assayed in a sample from the subject.

12. The method of claim 10, wherein the amount of at least one of ALT, ASP, and ALP are assayed in a sample from the subject.

13. The method of claim 10, wherein the score is determined using the algorithm:
Score=y/(1+y)
wherein,
y=exp [−X+(C₁×Age)+(C₂×Tr-L/p)+(C₃×ALT)+(C₄×Bilirubin)+(C₅×Albumin)]
wherein X is from 0.7274 to 16.3490 inclusive;
C₁ is from 0.0368 to 0.1682 inclusive;
C₂ is from 0.0494 to 0.1556 inclusive;
C₃ is from 0.0173 to 0.0433 inclusive;
C₄ is from 0.7512 to 3.9666 inclusive; and
C₅ is from −6.4955 to −2.6163 inclusive;
and wherein, age is in years: normalized Tr-L (Tr-L/p) is in pmol product /sec/pg proteasome; ALT is in IU/L and bilirubin and ubiquitin are in mg/dL.

14. The method of claim 13, wherein
X is about 8.5382;
C₁ is about 0.1025;
C₂ is about 0.1025;
C₃ is about 0.0303;
C₄ is about 2.3589; and
C₅ is about −4.5559.

15. The method of claim 13, wherein the reference score is about 0.5 and a score less than about 0.5 is indicative of the absence of advanced liver fibrosis in the subject.

16. The method of claim 13, wherein the reference score is about 0.5 and a score greater than or equal to about 0.5 is indicative of advanced liver fibrosis in the subject.

17. The method of claim 13, wherein the reference score is about 0.5 and a score less than about 0.5 is indicative of a Metavir fibrosis score of F0, F1 or F2 in the subject.

18. The method of claim 13, wherein the reference score is about 0.5 and a score greater than or equal to about 0.5 is indicative of a Metavir fibrosis score of F3 or F4 in the subject.

19. The method of claim 10, wherein the sample is serum or plasma.

20. The method of claim 10, wherein the score is used for the choice of a suitable treatment for the subject.

21. A method for diagnosing a chronic liver disease in a subject, the method comprising:
(a) determining the specific activity of one or more proteasomal peptidases selected from the group consisting of chymotrypsin-like activity (Ch-L), trypsin-like activity (Tr-L), and caspase-like activity (Cas-L) in an acellular body fluid from the subject, wherein the specific activity is determined by normalizing the one or more peptidase activities to the amount of proteasomal protein in the acellular body fluid from the subject;
(b) determining a single diagnostic score for the subject based on said determination; and,
(c) comparing the single diagnostic score for the subject to a reference score, wherein said comparing is used to determine a diagnosis a chronic liver disease in the subject.

22. The method of claim 21, wherein the chronic liver disease is hepatitis C virus (HCV)-related chronic liver disease.

23. The method of claim 21, wherein the acellular body fluid is selected from the group consisting of serum and plasma.

24. The method of claim 21, wherein the reference specific activity is the specific activity in a comparable acellular body fluid from one or more healthy individuals.

25. A method of diagnosing a chronic liver disease in a subject, the method comprising:
(a) determining the amount of proteasomal protein in a test sample from the subject;
(b) determining the level of one or more proteasomal peptidase activities in a test sample from the subject, the peptidase activities selected from the group consisting of chymotrypsin-like activity (Ch-L), trypsin-like activity (Tr-L), and caspase-like activity (Cas-L),
(c) normalizing the level of one or more proteasomal peptidase activities to the amount of proteasomal protein in the test sample to provide a specific activity of the one or more proteasomal peptidases;
(d) using the specific activity of the one or more proteasomal peptidases to generate a single diagnostic score; and
(e) comparing the single diagnostic score for the subject to a reference score to allow the diagnosis of a chronic liver disease in the subject.

26. The method of claim 25, wherein the chronic liver disease is hepatitis C virus (HCV)-related chronic liver disease.

27. The method of claim 25, wherein the test sample is an acellular body fluid sample.

28. The method of claim 27, wherein the acellular body fluid is selected from the group consisting of serum and plasma.

29. The method of claim 25, wherein the test sample is a cell-containing sample.

30. The method of claim 25, wherein the reference score is determined from one or more healthy individuals.
